(19) United States
(12) Patent Application Publication
(10) Pub. No.: US 2007/0196920 A1
(43) Pub. Date: Aug. 23, 2007

(54) Method and device of metamorphosing cells, and treatment apparatus using the same

(75) Inventors: Kazuhiro Suzuki, Minato-ku (JP); Yujiro Naruse, Yokohama-shi (JP); Hideyuki Funaki, Shinagawa-ku (JP); Kazuhiko Itaya, Yokohama-shi (JP); Shuichi Uchikoga, Yokohama-shi (JP)

Correspondence Address: OBLON, SPIVAK, MCCLELLAND, MAIER & NEUSTADT, P.C., 1940 DUKE STREET, ALEXANDRIA, VA, 22314, US

(73) Assignee: KABUSHIKI KAISHA TOSHIBA, Minato-ku (JP)

(21) Appl. No.: 11/507,674

(22) Filed: Aug. 22, 2006

(30) Foreign Application Priority Data
Dec. 2, 2005 (JP) 2005-349255

(51) Int. Cl. C12N 15/09; C12N 15/00; C12M 3/00

(52) U.S. Cl. 435/455; 435/285.3; 977/902

(57) ABSTRACT

A cell metamorphosing device includes micro dishes which serves as diaphragms and hold a mixed medium containing harmful cells and nano-scale particles, an AC voltage supply, a heater and an inductor. The AC voltage supply faces with the micro dishes 2 with a space, and applies a bias to the micro dishes 2, so that the nano-scale particles are bombarded onto the harmful cells and destroy them.

### CROSS REFERENCE TO RELATED APPLICATIONS

This application is based upon and claims the benefit of priority from prior Japanese Patent Application No. 2005-349255 filed on Dec. 2, 2005, the entire contents of which is incorporated by reference herein.

### BACKGROUND OF THE INVENTION

1. Field of the Invention

This invention relates to a method and a device of metamorphosing cells, and a treatment apparatus using the same, and more particularly relates to a method, a device, and a treatment apparatus which are applicable to metamorphosing cells harmful to living bodies.

2. Description of the Related Art

Biochemical antibiotics like Penicillin, Vancomycin and Methicillin are very effective in curing bacterial diseases such as pneumonia and blood poisoning excited by viruses or bacteria. However, the foregoing antibiotics produce side effects, and use of antibiotics results in multiple drug-resistant bacteria, aftereffects caused by drug interaction, and so on. Further, some antibiotics are effective to bacterium but not effective to viruses.

JP-A-2005-102619(Kokai) (called the “Reference 1”) discloses a device which is used to effectively change structures, conditions or functions of cells extracted from a living body. The device includes a medium container which houses a fluid containing cells, and a mechanical vibrator which vibrates a transducer. The vibrations have a specific frequency which depends upon the mass of the transducer and a frictional attenuation coefficient of the fluid. Specifically, to change the cell structure means to extract DNA (deoxyribonucleic acid) from nuclei of cells, to perform gene recombination, to infuse artificially produced RNA (ribonucleic acid) or protein into a cell membrane, to control cell division, to improve resistance to viruses, and so on.

JP-A-2004-290351(Kokai) (called the Reference 2″) describes a cancer treatment apparatus. Ligands made of antibiotics which are selectively coupled to cancer cells are stuck on heating elements. Electromagnetic waves are applied onto the heating element in order to induction-heat the heating elements, so that only tumor cells or cancer cells are selectively heated and are broken down.

However, there is a concern that side effects may be caused by biochemical antibiotics administered to the living body, and that there is a chance of multiple drug resistant bacteria. Therefore, it is inevitable to develop and administer new antibiotics to the living body.

Reference 1 describes, in Field of the Invention, to distinguish viruses from cells, to break down viruses, and to effectively change the structure, state, functions and so on of cells. However, it does not specifically describe how to effectively destroy cancer cells.

Reference 2 describes that heating elements are adsorbed onto walls of cancer cells or cell membranes in order to destroy cells. However, since some cancer cells are resistant to heat, it is very difficult to effectively destroy all of the cancer cells.

### SUMMARY OF THE INVENTION

The present invention has been contemplated in order to overcome problems of the related art, and is intended to provide a cell metamorphosing method and a cell metamorphosing device which transform and destroy cells harmful to living bodies, and a treatment apparatus constituted by the cell metamorphosing device.

According to a first aspect of the embodiment of the invention, there is provided a cell metamorphosing method which includes mixing nano-scale particles in a medium containing harmful cells, and applying vibration energy and thermal energy to the nano-scale particles, bombarding the nano-scale particles to the harmful cells, and destroying the harmful cells.

In accordance with a second aspect of the embodiment, there is provided a cell metamorphosing device which includes a medium container housing a mixed medium containing harmful cells and nano-scale particle, a vibrator vibrating the mixed medium, and a heater heating the mixed medium.

According to a final aspect of the embodiment, there is provided a treatment apparatus which includes an endoscope, a light source supplying light to the endoscope, a first drive unit coupled to the endoscope and activating a leading end of the endoscope, and a treatment section attached to the leading end of the endoscope, and including a cell metamorphosing device and a second drive unit. The cell metamorphosing device includes a medium container housing a medium containing harmful cells and nano-scale particles, a vibrator applying vibrations to the mixed medium, and a heater heating the mixed medium. The second drive unit moves the cell metamorphosing device in an imaging direction of the endoscope.

### BRIEF DESCRIPTION OF THE DRAWINGS

FIG. 1 is a perspective view of an essential part of a cell metamorphosing device according to the embodiment of the invention;

FIG. 2 is a perspective view showing an overall structure of the cell metamorphosing device;

FIG. 3 is a sectional view of the essential part of the cell metamorphosing device of FIG. 1, taken along line F3-F3 shown in FIG. 1;

FIG. 4A is a graph showing the relationship between forward currents of a heating element and an increase of temperature of a micro dish;

FIG. 4B is a graph showing the relationship between a forward current of an inductor and a magnetic flux density of the heating elements;

FIG. 5 shows the structure of a eukaryotic cells and an induced state of a nano-scale particle, in order to explain the cell metamorphosing method;

FIG. 6 shows the structure of a procaryotic cell and an induced state of a particle, in order to explain the cell metamorphosing method;

FIG. 7 schematically shows the cell metamorphosing device, explaining the cell metamorphosing method;

FIG. 8A shows a model of a nano-scale particle in a mixed medium;

FIG. 8B shows the relationship between a flow rate of a thermal boundary layer in the mixed medium and a temperature;

FIG. 9 is a sectional view of the cytomorphorsis device in a first manufacturing step;

FIG. 10 is a sectional view of the cell metamorphosing device in a second manufacturing step;

FIG. 11 is a sectional view of the cell metamorphosing device in a third manufacturing step;

FIG. 12 is a sectional view of the cell metamorphosing device in a fourth manufacturing step;

FIG. 13 is a sectional view of the cell metamorphosing device in a fifth manufacturing step;

FIG. 14 is a conceptual diagram showing a further cell metamorphosing method;

FIG. 15A is a conceptual diagram of a treatment apparatus according to the embodiment of the invention; and

FIG. 15B is an enlarged view of an essential part of the treatment apparatus.

### DETAILED DESCRIPTION OF THE INVENTION

Following is a detailed description of the invention as illustrated by the attached drawings, in which like numerals refer to like part throughout. The drawings are schematic, and are depicted using scales which sometimes differ from those of actual products, and which are different in some drawings.

[CELL METAMORPHOSING DEVICE]

A cell metamorphosing device of the embodiment of the invention includes a medium container, a vibrator, and a heater. The medium container houses a mixed medium in which nano-scale particles are mixed with harmful cells. The vibrator vibrates the mixed medium. The heater heats the mixed medium.

Harmful cells adversely affect living bodies, and are classified into eukaryotic cells which have nucleuses and form a chromosome structure at the time of cell division, and procaryotic cells which do not have structurally discernible nucleuses. Specifically, harmful cells are cancer cells, tumor cells (eukaryotic cells) and so on extirpated from living bodies, and procaryotic cells such as viruses and bacteria.

For instance, nano-scale particles are minute particles whose diameters are 10 nm to 100 nm, and are preferably gold (Au), silicon (Si), or the like.

The medium container houses the mixed medium, and may have any structure so long as the mixed medium can be vibrated, be heated, be applied a magnetic field, and so on. The medium container is made of micro dishes, a silicon substrate, a quartz substrate, or the like.

The medium container preferably has its inner surface covered by a water-repellant film. The mixed medium is made to drop into the medium container. The medium container can remain reliable in a wet environment without being electrically short-circuited, and can be stably and reliably excited by electrostatic power.

The vibrator may be structured as desired so long as it can apply vibration energy to the mixed medium. The vibration energy vibrates nano-scale particles from right to left, and up and down.

The vibrator is preferably a voltage supply which is placed apart from the medium container. An AC bias is applied between the medium container and the voltage supply, thereby vibrating the medium container, and applying the vibration energy to nano-scale particles.

The heater may be designed or structured as desired so long as it can apply thermal energy to the mixed medium. The thermal energy promotes vibrations of nano-scale particles heated by the heater, so that nano-scale particles generate energy.

The cell metamorphosing device vibrates nano-scale particles and heats them, which enables nano-scale particles to vibrate in a specified direction at an increased speed, to hit harmful cells with a strong acceleration force, and to destroy harmful cells.

The cell metamorphosing device is preferably provided with a magnetic field applicator which applies a magnetic field to the mixed medium. The magnetic field applicator may be designed or structured as desired so long as it can apply the magnetic field. An inductor is used as the magnetic field applicator, for instance. The magnetic field is applied to the mixed medium in order to control a direction of nano-scale particles accelerated and heated by the vibrator and the heater. Therefore, more nano-scale particles can reliably hit harmful cells, and destroy them.

Further, the cell metamorphosing device preferably includes an electromagnetic wave applying unit which applies electromagnetic waves to the mixed medium. The electromagnetic wave applying unit may be designed or structured as desired so long as it can emit electromagnetic waves. For instance, the electromagnetic wave applying unit may be constituted by light emitting elements. Therefore, higher thermal energy (optical energy) can be applied to nano-scale particles which are accelerated by the vibrator and the heater, so that nano-scale particles can hit harmful cells at an accelerated speed and destroy them effectively.

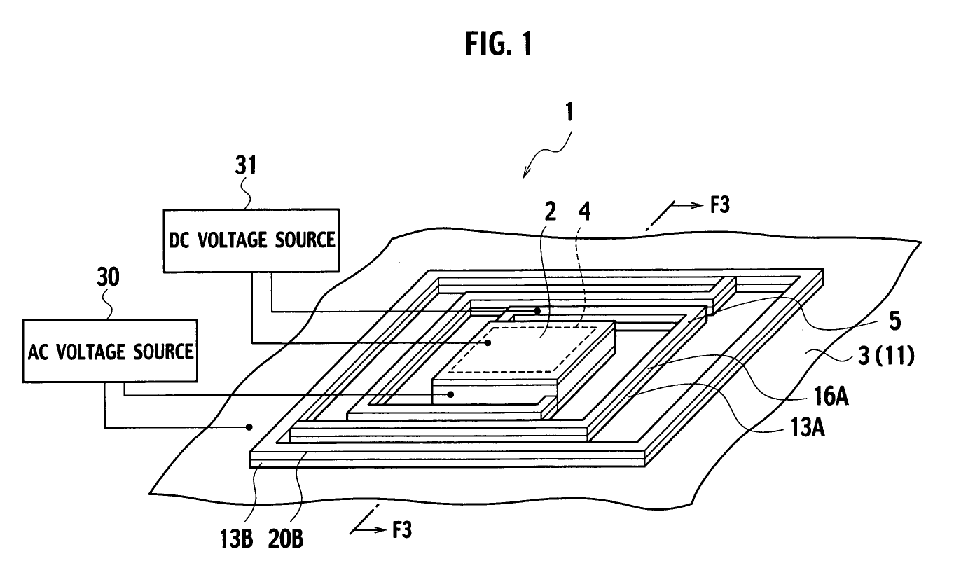

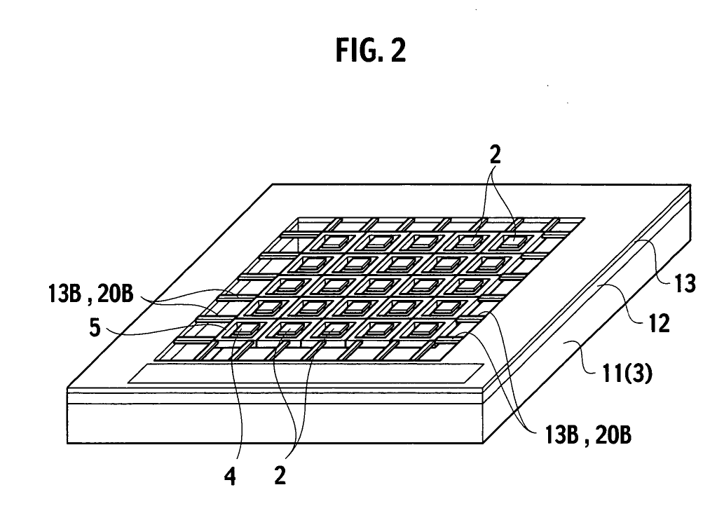

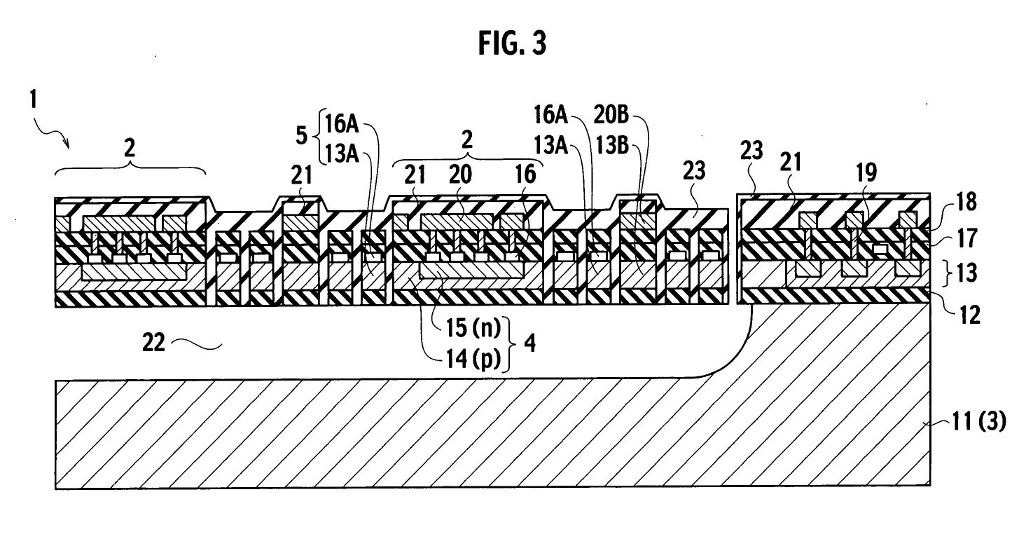

Referring to FIG. 1 to FIG. 3, the cell metamorphosing device 1 includes a plurality of micro dishes 2, and a substrate 11. The micro dishes 2 are used as diaphragms, and retain thereon mixed medium containing harmful cells and nano-scale particles. The substrate 11 functions as a voltage supply, and is placed apart from the micro dishes 2. In other words, the micro dishes 2 are placed in rectangular cavities 22 which are regularly formed on the substrate 11, and are arranged in the shape of a matrix. In FIG. 2 and FIG. 3, each micro dish 2 is identically structured and shaped.

The micro dishes 2 and the substrate 11 are connected to an AC power supply 30. The cell metamorphosing device 1 further includes a vibrator which vibrates the micro dishes 2 when an AC bias is applied between the micro dishes 2 and the substrate 11.

As shown in FIG. 3, each micro dish 2 includes a compartmentalized insulator 12; an active layer 13 on the compartmentalized insulator 12; a first wiring 16 on the active layer 13; interlayer dielectrics 17 and 18 on the first wiring 16; a second wiring 20 on the interlayer dielectric 18; a protective film 21 on the second wiring 20; and a water-repellent film 23 coated on the protective film 21.

Each micro dish 2 is held in position by first mechanical supports 13A and 16A which are mechanically coupled to the micro dishes 2. The first mechanical supports 13A and 16A are joined to second mechanical supports 13B and 20B. The second mechanical supports 13B and 20B are placed on the periphery of the substrate 11.

In each micro dish 2, the first mechanical support 13A is integral with the active layer 13 while the mechanical support 16A is integral with the first wiring 16. The second mechanical supports 13B and 20B are structured similarly to the mechanical supports 13A and 16A. The second mechanical support 13B is integral with the active layer 13, and the second mechanical support 20B is integral with the second wiring 20.

Each micro dish 2 is joined to the periphery of the substrate 11 via the first mechanical supports 13A and 16A, and the second mechanical supports 13B and 20B, and functions as a mechanical diaphragm which can be excited (can be vibrated) vertically and laterally. When an AC bias is applied to the substrate 11 and each micro dish 2 from the AC power supply 30, an electrostatic force is generated, and enables each micro dish 2 to vibrate vertically and laterally on the substrate 11.

In the active layer 13 on the micro dish 2, a diode (a heater 4) is formed by not only an anode region 14 which is made by implanting or diffusing p-type impurities but also by a cathode region 15 which is made by implanting or diffusing n-type impurities onto the surface of the anode region 14. The anode region 14 and the cathode region 15 are joined by the p-n junction. The anode region 14 is mechanically and electrically connected to the mechanical support 13A, which is connected to a DC power supply 31 placed outside the substrate 11. Therefore, a forward current flows to the anode region 14.

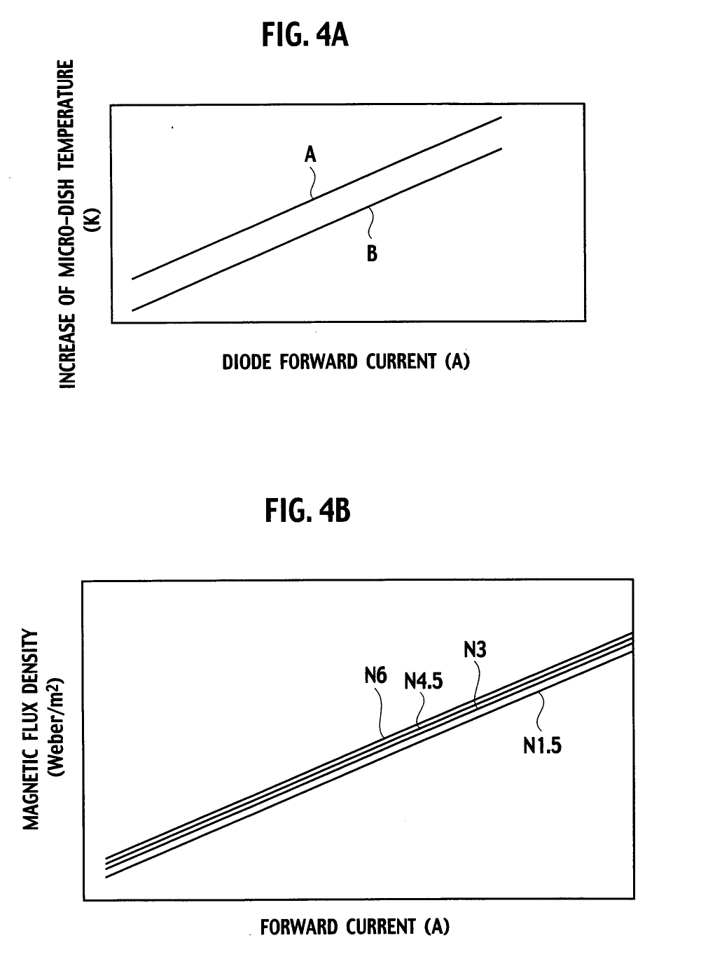

The cathode region 15 is electrically connected to the first wiring 16 which is placed on the cathode region 15. The first wiring 16 is electrically connected to a second wiring 20 via a wiring 19. The second wiring 20 is electrically connected to the DC power source 31, and feeds the supplied forward current back to the anode region 14. In short, when the forward current flows, the active layer 13 functions as a heater and locally heats the micro dishes 2. The active layer 13 is hereinafter referred as the “heater 4”. As shown in FIG. 4A, the heater 4 heats the micro dishes 2 in response to the application of the forward current. In FIG. 4A, the line A denotes a temperature rise when the forward current is applied to infinity while the line B denotes a temperature rise when the forward current is applied for one millisecond (1 msec).

The first mechanical supports 13A and 16A extend around the micro dishes 2 with a specified space maintained, and function as an inductor 5. Although not shown in detail, the second mechanical support 16A is positioned on the first mechanical support 13A, and the mechanical supports 13A and 16A are electrically connected. The second mechanical support 13B is provided via the interlayer dielectrics 17 and 18, and is electrically connected to the interlayer dielectrics 17 and 18 via connection holes therein. When the heater 4 is activated, the forward current flows to the first mechanical supports 13A and 16A. Therefore, a magnetic field is produced around the first mechanical supports 13A and 16A, and reaches the micro dishes 2. In this example, the first mechanical support 13A joined to one end of the micro dishes 2 extends around three sides of the micro dish 2 in order that the inductor 5 has approximately 1.5 turns. FIG. 4B shows the relationship between magnetic flux densities and the number of turns. In FIG. 4B, N1.5 denotes 1.5 turns, N3 denotes 3 turns, N4.5 denotes 4.5 turns, and N6 denotes 6 turns. Fundamentally, the number of turns makes little difference. The magnetic flux density becomes higher in proportion to the forward current.

The substrate 11 is preferably a semiconductor substrate, and more specifically a silicon single crystal substrate. The insulator 12 is preferably a silicon oxide film, and serves as a buried oxide layer (BOX). The active layer 13 is preferably a semiconductor active layer, and more specifically a single or polycrystalline crystal silicon layer. In short, the cell metamorphosing device 1 is constituted by an SOI (silicon-on-insulator) substrate which includes the substrate 11, insulator 12 and active layer 13. The cell metamorphosing device 1 has a square or rectangular planar shape. Alternatively, the planar shape of the cell metamorphosing device 1 may be circular, oval or polygonal.

The first wiring 16 is preferably a gate material used as an electrode of a passive or active component, specifically a polycrystalline silicon film, a compound film made of silicon and a refractory metal, a single layer of a refractory metal, or a composite lamination containing the foregoing compound film or the refractory metal.

The second wiring 20 is made of a metal whose resistance is lower than that of the first wiring 16, and more specifically an aluminum alloy film, which contains silicon (Si) in order to suppress alloy spikes or copper (Cu) in order to electro-migration.

The first wiring 16 and the second wiring 20 are electrically connected via a connection hole wiring 19 placed in the interlayer dielectrics 17 and 18. The interlayer dielectrics 17 and 18 and the protective film 21 are preferably oxide silicon films, nitride silicon films or compound films of oxide or nitride silicon films.

The water-repellent film 23 is made of water-repellent silicone or the like, increases a contact angle of the medium dripping thereon, and vibrates medium dripping onto the micro dishes 2. The water-repellent film 23 is made by placing the cell metamorphosing device 1 in a mixed ambient gas containing C₈F₁₃H₄SiC₃ gas and H₂O gas.

Each micro dish 2 is a square whose one side is 20 μm long. Each micro dish 2 and the first and second supports 13A and 16A (and the inductor 5) supporting the micro dish 2 are placed on an area whose one side is 30 μm long. The micro dishes 2 are arranged in the shape of 30 μm check boards. Alternatively, the micro dishes 2 may be sized as desired. The length of 30 μm corresponds to each space between adjacent micro dishes 2.

[FIRST CELL METAMORPHOSING METHOD]

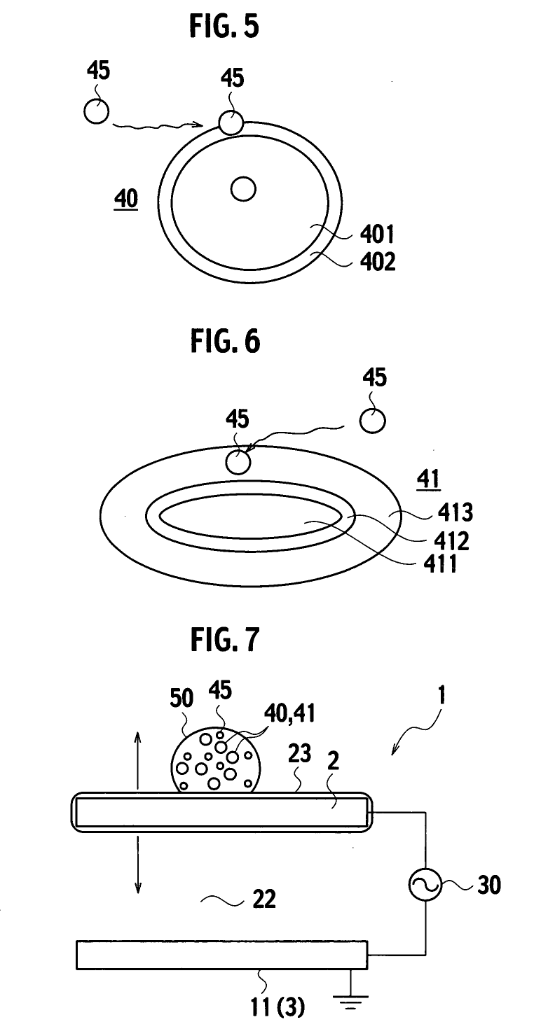

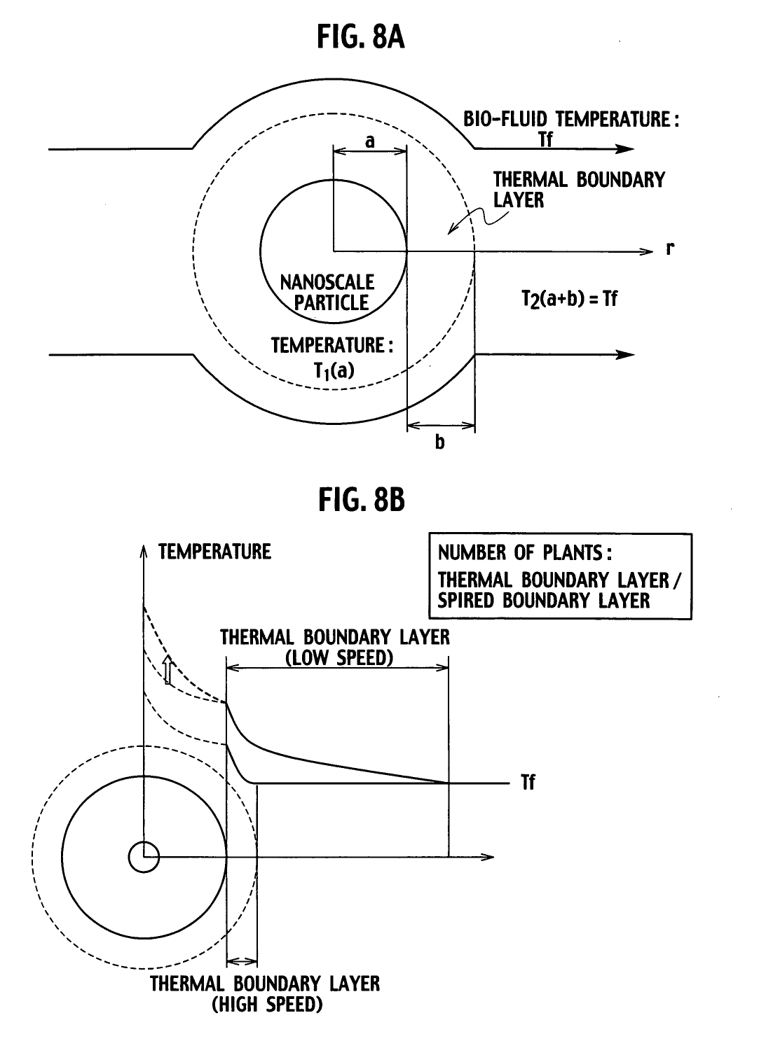

A first cell metamorphosing method will be described with reference to FIG. 5, FIG. 6, FIG. 7, FIG. 8A and FIG. 8B.

According to this invention, cells are metamorphosed by mixing nano-scale particles in the medium containing harmful cells, applying vibration energy and thermal energy to the nano-scale particles, making the nano-scale particles strike harmful cells, and physically destroying harmful cells.

Harmful cells adversely affect living bodies, and are classified into eukaryotic cells which have nucleuses and form a chromosome structure at the time of cell division, and procaryotic cells which do not have structurally discernible nucleuses. Specifically, harmful cells are cancer cells, tumor cells (eukaryotic cells) and so on extirpated from living bodies, and procaryotic cells such as viruses and bacteria.

Referring to FIG. 5, a eukaryotic cell 40 includes a cell membrane 402 extending over a cytoplasm 401, for example. As shown in FIG. 6, a procaryotic cell 41 includes a cell membrane 412 covering a cytoplasm 411, and a cell wall 413 covering the cell membrane 412.

The nano-scale particles 45 are minute, have a grain size of 10 nm to 100 nm, and are preferably gold (Au), silicon (Si) and so on. Vibration energy and thermal energy are preferably applied to nano-scale particles on the water-repellent film 23. The water-repellent film 23 can prevent a short-circuit in a wet ambient, and effectively apply the vibration and thermal energy to the nano-scale particles.

The nano-scale particles 45 applied with the vibration energy and thermal energy pass through harmful cells or get into harmful cells, and destroy harmful cells.

The vibrator vibrates the medium container in order to apply vibration energy to nano-scale particles 45 in the mixed medium while the heater heats nano-scale particles in the medium container. When the mixed medium is heated, nano-scale particles 45 are selectively and precisely induced to the cell membrane 402 of the cell 40 or the cell wall 413 of the cell 41, as described in detail hereinafter.

As shown in FIG. 6, the cell wall 413 of the cell (the procaryotic cell) 41 is made of peptide glycan having a molecular architecture in which tetra peptide and penta glycine lap pile up on sugar chains. The cell wall 413 having the foregoing molecular architecture abruptly changes its state to “adsorption or non-adsorption” at a transition temperature of glass. As a temperature of the nano-scale particles 45 passing through the cell 41 is raised due to frictional heat, members surrounding the nano-scale particles 45 are also heated, which causes the cell wall 413 to change its state to the “adsorption”. Therefore, nano-scale particles 45 are selectively guided to the cell wall 413, and strike the cell wall 413. In this case, nano-scale particles 45 pass through the cell wall 413 or are taken in the cell wall 413. Refer to FIG. 5 and FIG. 6.

The temperature of the nano-scale particles 45 is precisely controlled on the basis of an equation of heat conduction, as will be described with reference to a model shown in FIG. 8A.

Temperature distribution T₁(r) in the nano-scale particles 45 which are spherically symmetrical is expressed by a formula (1).
T₁(r)=A/r+B, 0<r<a (1)

Where parameters A and B are unknown quantities and are optional, and a parameter a denotes a semi diameter of the nano-scale particles 45.

Temperature distribution T₂(r) of a thermal boundary layer (stagnant layer) on the nano-scale particles 45 is expressed by a formula (2).
T₂(r)=C/r+D, a<r<a+b (2)

Where a parameter b denotes a width of a stagnant layer in the mixed medium 50, and parameters C and D denote arbitrary constants.

According to the theory of thermal conduction, a total amount of energy generated in the nano-scale particles 45 (i.e., the left-hand side) is equal to a total amount of energy running off from the surfaces of the nano-scale particles 45 (i.e., the right-hand side), as expressed by a formula (3). In the formula (3), the left-hand side denotes a value derived by multiplying a temperature gradient of the surface of the nano-scale particles 45, a surface area and a coefficient λ a of thermal conductivity. The right-hand side denotes a value derived by multiplying an energy generating ratio g of a magnetic field (unit bulk/unit time) and the bulk of the nano-scale particles 45.
−λa(dT₁/dr) r=a·4πa2=4πa3·g/3 (3)

A function formula T₂ for temperature distribution of a thermal boundary area expresses a physical phenomenon similarly to the formula (3), but should be also established when r=a. The formula (3) can be established when r=a in the temperature distribution T₁ in the nano-scale particles 45. A total amount of energy generated in the nano-scale particles 45 can be expressed by a formula (4) for an area outside the nano-scale particles 45 when a coefficient of λₕ of thermal conductivity of the mixed medium 50.
−λₕ(dT₂/dr) r=a·4πa2=4πa3·g/3 (4)

A flow temperature Tf is used as a boundary condition expressed by a formula (5) for an area outside the thermal boundary layer.
T₂(a+b)=Tf (5)

The coefficients T₁ and T₂ should be continuous when r=a, as expressed by a formula (6).
T₁(a)=T₂(a) (6)

By making the formulas (3) to (6) into a simultaneous equation, a temperature T₁ (a) of the nano-scale particles 45 can be derived as expressed by a formula (7).
T₁(a)=Tf+(a2g/3λh)·b/(a+b) (7)

FIG. 8B shows the relationship between a flow rate and a temperature of the thermal boundary layer based on the formula (7). The nano-scale particles 45 are caught by the cell 40 or 41 (i.e., the nano-scale particles 45 strike on the cell wall 402 or 413 and break therein). If the flow rate of the mixed medium 50 is low around the nano-scale particles 45, the thermal boundary layer is generated on the nano-scale particles 45, and a rate of temperature rise is increased between the thermal boundary layer and the nano-scale particles 45. Therefore, the mixed medium 50 around the nano-scale particles 45 is heated, so that the cell membrane 402 of the cell 40 or the cell wall 413 of the cell 41 is solved (is subject to physical impact due to heat), and the cell 40 or 41 will be destroyed. Further, a quick thermal expansion of the nano-scale particles 45 damages the cell membrane 402 of the cell 40 or the cell wall 413, which will destroy the cell 40 or 41. Still further, the cell 40 or 41 will blow itself up and be destroyed due to an inner pressure (e.g., 20 Pa) of a cellular cytoplasm 401 of the cell 40 or a cellular cytoplasm 411 of the cell 41.

Receiving the vibration energy and thermal energy, the nano-scale particles 45 can destroy the cell 40 or 41. For instance, when the DC current is supplied to the heater 4 (diode) of the cell metamorphosing device 1 from the DC power supply 31, the micro dishes 2 are heated, which effectively and extensively promotes the acceleration of the nano-scale particles 45. Therefore, the nano-scale particles 45 can shoot out the cell membrane 402 and the cell wall 413, or the nano-scale particles 45 can get into the cell membrane 402 and the cell wall 413. Even when the nano-scale particles 45 have gotten into the cell membrane 402 and the cell wall 413 as shown in FIG. 5 and FIG. 6, the temperature of the mixed medium 50 around the nano-scale particles 45 is raised, and the nano-scale particles 45 are extensively expanded, which are effective in destroying the cell 40 or 41.

Further, it is preferable to apply magnetic field energy to the nano-scale particles 45. For instance, as soon as a DC voltage is applied to the heater 4 of the cell metamorphosing device 1 shown in FIG. 2, a DC current is simultaneously applied to the inductor 5, which will generate a magnetic field in the mixed medium 50. Therefore, the nano-scale particles 45 are further accelerated, bump against the cell membrane 402 or the cell wall 413, and increase forces to pass through the cell membrane 402 or the cell wall 413. Further, the nano-scale particles 45 adjust themselves to the magnetic field, are accelerated in a preset direction, and bump against the cell membrane 401 or the cell wall 413 more frequently, and more reliably destroy the cell 40 or 41.

### EXAMPLE 1

Referring to FIG. 7, first of all, the mixed medium 50 is prepared by adding the nano-scale particles 45 into a solution containing the harmful cell 40 or 41. The mixed medium 50 is dropped onto micro dishes 2 of the cell metamorphosing device 1.

An AC voltage is applied to the substrate 11 from the AC power supply 30, so that an AC bias will be applied between the substrate 11 and the micro dishes 2. Therefore, the micro dishes 2 are vibrated vertically and horizontally with respect to the surface of the substrate 11 (as shown in FIG. 7), which applies the vibration energy to the nano-scale particles 45.

Further, the DC power source 31 supplies the DC current to the heater 4 of the cell metamorphosing device 1 in order to heat the micro dishes 2, so that the nano-scale particles 45 will receive the thermal energy and electric field energy.

The mixed medium 50 is collected, and is observed using an optical microscope in order to check states of the harmful cell 40 or 41. It is confirmed that the nano-scale particles 45 have got into the cell membrane 402 or the cell wall 413 as shown in FIG. 5 and FIG. 6.

### COMPARISON EXAMPLE 1

First of all, a mixed medium 50 is prepared by adding the nano-scale particles 45 into a solution containing the harmful cell 40 or 41 as shown in FIG. 7. The mixed medium 50 is made to drop onto the micro dishes 2 of the cell metamorphosing device 1.

The AC voltage is applied to the substrate 11 from the AC power supply 30, so that the AC bias is applied between the substrate 11 and the micro dish 2. Therefore, the micro dishes 2 are vibrated vertically and horizontally with respect to the surface of the substrate 11, which applies the vibration energy to the nano-scale particles 45 as shown in FIG. 7.

Without heating the micro dishes, the mixed medium 50 is collected, and is observed using an optical microscope in order to check states of the harmful cell 40 or 41. No change is observed in the harmful cell 40 or 41.

### COMPARISON EXAMPLE 2

First of all, a mixed medium 50 is prepared by adding the nano-scale particles 45 into a solution containing the harmful cell 40 or 41 as shown in FIG. 7. The mixed medium 50 is dropped onto the micro dishes 2 of the cell metamorphosing device 1.

No AC voltage is applied to the substrate 11. However, the DC current is applied to the heater 4 of the cell metamorphosing device 1 from the DC power supply 31 in order to heat the micro dishes 2. This applies the thermal energy and electric field energy to the nano-scale particles 45.

The mixed medium 50 is collected, and is observed using an optical microscope in order to check states of the harmful cell 40 or 41. No change is observed in the harmful cell 40 or 41.

[METHOD OF FABRICATING CELL METAMORPHOSING DEVICE]

The cell metamorphosing device 1 is fabricated as shown in FIG. 1 to FIG. 3.

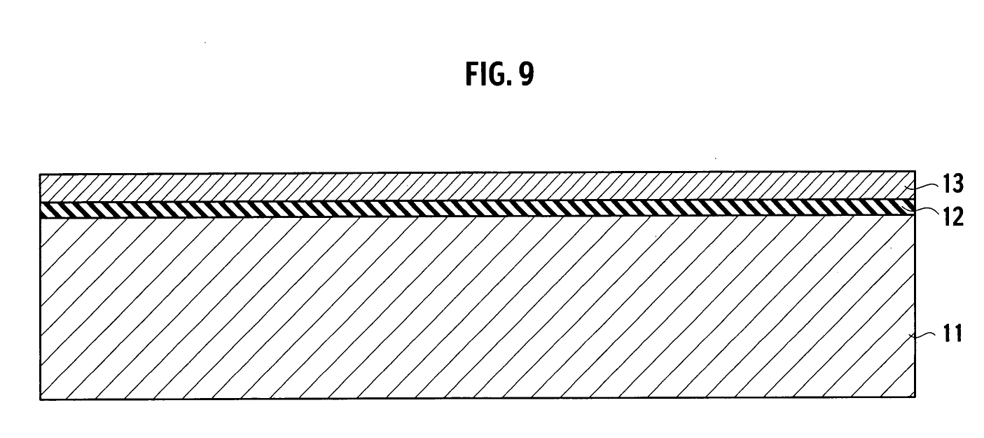

Referring to FIG. 9, an SOI substrate in which the substrate 11, insulator 12 and active layer 13 are piled is prepared. The SOI substrate is fabricated by implanting oxide ions into a silicon single crystal substrate from its front surface, and the insulator 12 is placed at a specified depth position of the silicone single crystal substrate. Alternatively, the SOI substrate may be prepared by a pasting process.

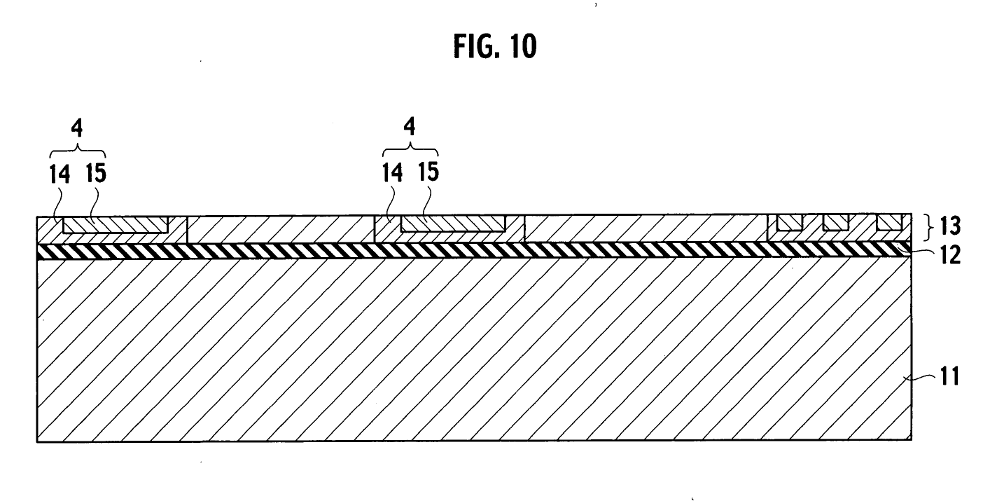

As shown in FIG. 10, p-type impurities are poured into specified positions of the active layer 13 where the micro dishes 2 are made, thereby making anode regions 14. A diode is made when the anode regions 14 and cathode regions 15 are made, and serves as the heater 4.

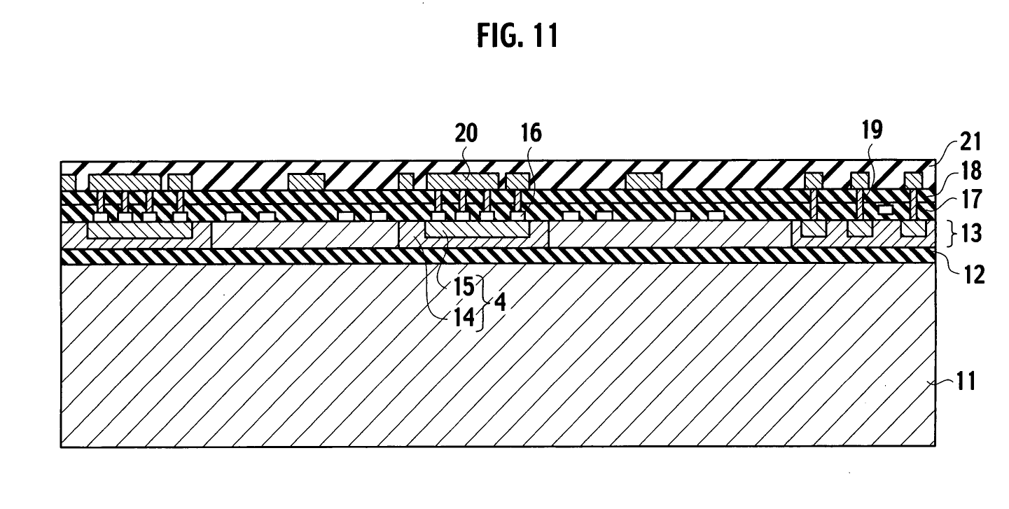

Thereafter, a first wiring 16, inter-layer dielectrics 17 and 18, a via hole wiring 19, a second wiring 20 and a protective film 21 are made on the heater 4 (active layer 13) one after another as shown in FIG. 11.

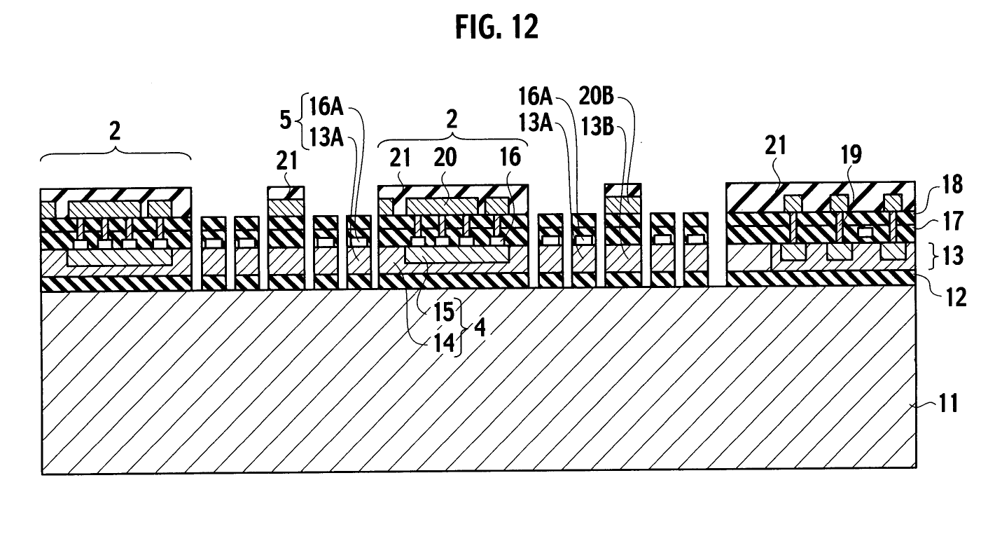

As shown in FIG. 12, the members extending over the substrate 11 are patterned by the photolithographic process and the etching process, so that first mechanical supports 13A and 16A, and second mechanical supports 13B and 20B are made. In this case, the protecting film 21 to the insulator 12 of the SOI substrate is used as an etching stop. The etching process is preferably the reactive ion etching (RIE). The inductor 5 is also made when the mechanical supports 13A and 16A are made.

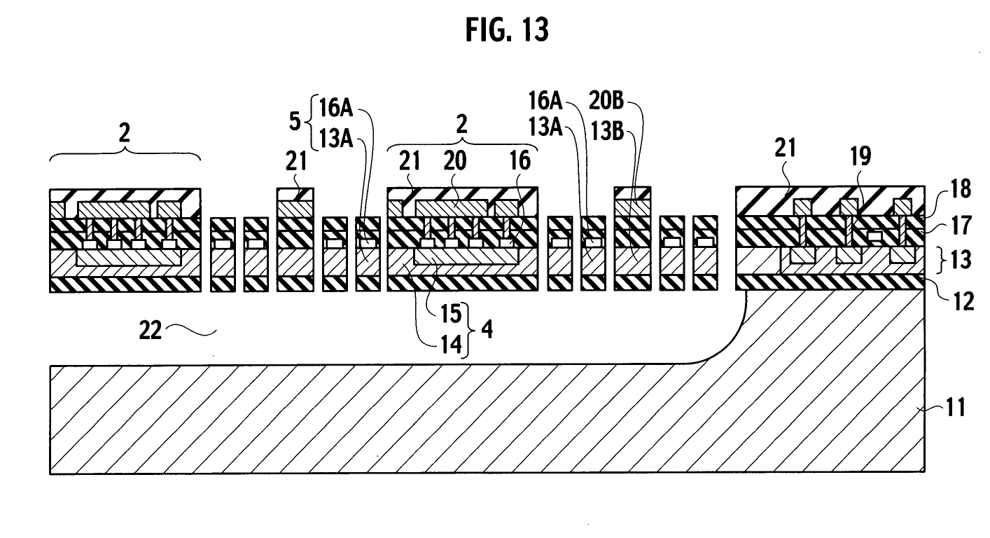

As shown in FIG. 13, the surface of the substrate 11 where a plurality of micro dishes 2 (where a center area of the substrate 11) are arranged is etched in order to form a cavity 22. The isotropic etching process is preferably performed using XeF₂ gas or an anisotropic etchant (KOH, THAH or the like).

As shown in the described above FIG. 3, the surfaces of the micro dishes 2 are covered by a water-repellent film 23. The cell metamorphosing device 1 will be completed after the foregoing processes.

[SECOND CELL METAMORPHOSING METHOD]

A second cell metamorphosing method is a modification of the cell metamorphosing device 1 and the first cell metamorphosing method.

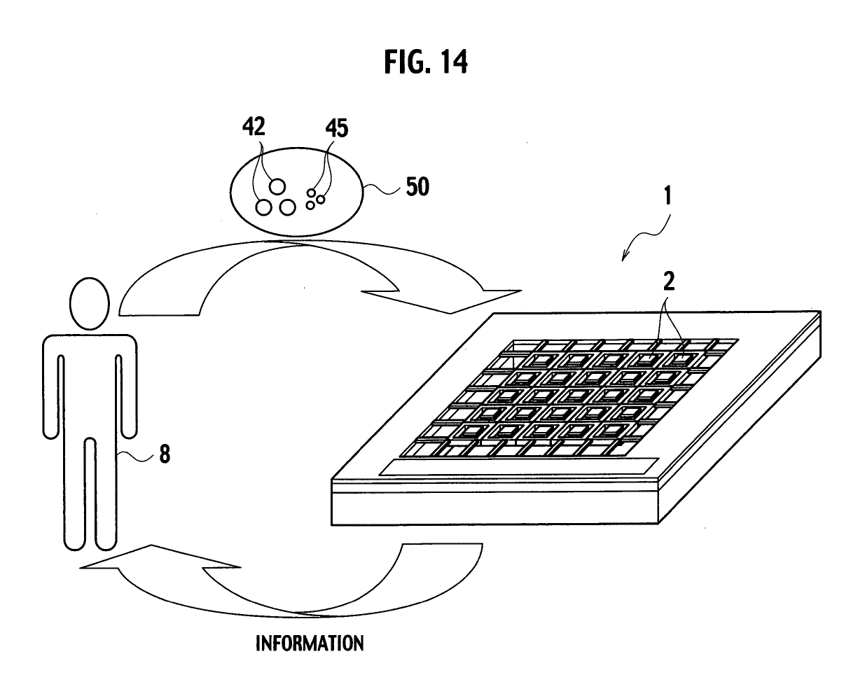

First of all, in FIG. 14, a harmful cell 42 is extracted from a living body 8, e.g., a patient. The harmful cell 42 denotes not only cancer cells, tumors, lesions or the like but also viruses, bacteria and so on which are not always extracted from the living body 8 but from cats, dogs, plants and so on.

The mixed medium 50 is prepared by applying the nano-scale particles 45 into the extracted harmful cell 42. This process is similar to the first cell metamorphosing method. Thereafter, the nano-scale particles 45 are made to strike onto the harmful cell 42 and destroy it. In this state, information for destroying the harmful cell 42 is acquired. Specifically, the information concerning the harmful cell 42 is collected and checked with respect to kinds and quantity of nano-scale particles 45, conditions for vibrating the micro dishes 2, heating conditions of the heater 4, conditions for generating magnetic force for the inductor 5, and so on.

The nano-scale particles 45 are injected into or are dosed to the living body 8. The cell metamorphosing device 1 is brought into contact with the living body 8. The cell metamorphosing device 1 is operated in accordance with information related to the harmful cell 42, so that the harmful cell 42 can be destroyed in the living body 8.

The invention offers the cell metamorphosing method in which the harmful cell 42 in the living body 8 is destroyed and is basically wiped out by applying physical impacts without causing side effects, and without generating bacteria which are resistant to a number of medical agents.

The cell metamorphosing device 1 has a simple structure, and includes the mechanical diaphragms, substrate 11 serving as the AC voltage supply 3, heater 4 realized by the diode, first mechanical supports 13A and 16A for the diaphragm, and inductor 5 serving as the heater 4. The cell metamorphosing device 1 can apply the vibration energy, thermal energy and magnetic field energy at the same time. The cell metamorphosing device 1 can generate mechanical vibrations, heat the nano-scale particles 45 by applying Joule heat, and produce a magnetic field. The cell metamorphosing device 1 is compatible with various kinds of cell destroying mechanisms and cell destroying conditions, and is applicable to a variety of tailored medical cares.

The invention is also applicable in the following situation. An MRI (Magnetic Resonance Imaging) or an optical sensor is used to locate lesions of harmful bacteria which are active in the living body 8 (i.e., patient). Then, the cell metamorphosing device 1 is brought into contact with the affected area of the patient who has taken nano-scale particles 45, in accordance with a recipe which is prepared to destroy the harmful cell at a clinical level. Therefore, the cell metamorphosing device 1 is applicable to curing diseases.

Further, it is possible to make a minute cell metamorphosing device 1, which includes a micro dish 2, a heater 4 and an inductor 5, and which can be taken into the living body 8. After locating a lesion of harmful bacteria which is present in the patient 8, the patient takes the nano-scale particles 45. Thereafter, the minute cell metamorphosing device 1 in the living body 8 is moved to the located lesion, so that the harmful cell 42 can be destroyed by the nano-scale particles 45. The minute cell metamorphosing device 1 is taken into the living body 8 and is guided to the lesion. Alternatively, the minute cell metamorphosing device 1 may be placed in the living body 8 by an operative surgery, and be guided to the lesion. A micro battery is applicable to the minute cell metamorphosing device 1 in order to supply power to the device 1. Further, the inductor 5 may be used as an antenna in order to generate power in response to electric waves.

[TREATMENT APPARATUS USING CELL METAMORPHOSING DEVICE]

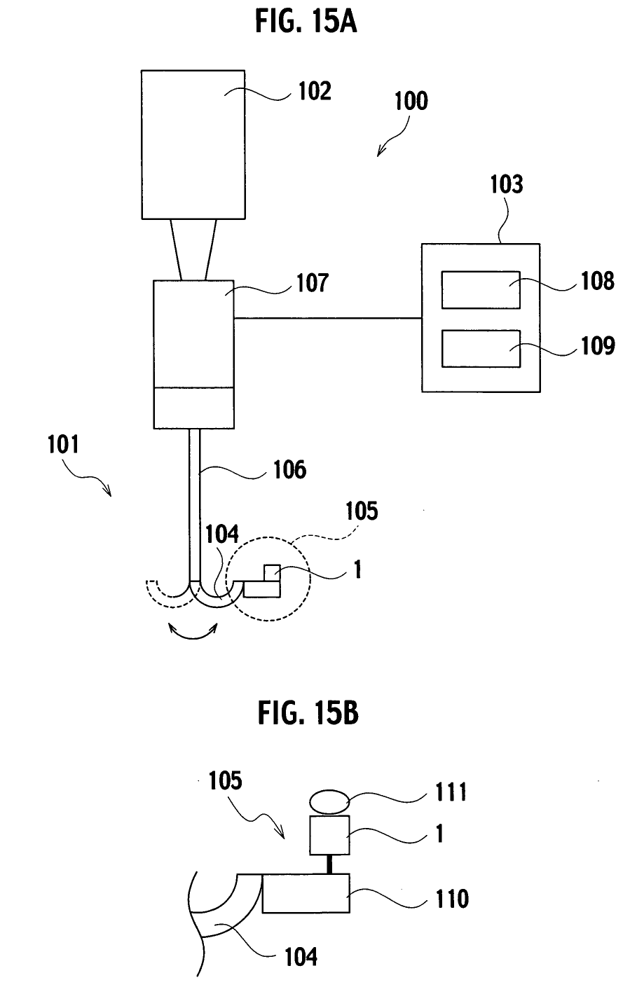

Referring to FIG. 15A and FIG. 15B, a treatment apparatus 100 is constituted by the cell metamorphosing device 1, an endoscope 101 including a solid-state image sensor (not shown), an optical source 102 supplying light to the endoscope 101, a controller 103 connected to the endoscope 101, and a treatment part 105 located at a leading end 104 of the endoscope 101.

The leading end 104 is curved and is connected to a body 107 of the endoscope 101 via a flexible part 106.

The controller 103 includes a display 108, and a first drive unit (not shown). The display 108 indicates images taken by the leading end 104 of the endoscope 101. The first drive unit I moves the curved leading end 104 up and down, or right to left.

The treatment part 105 is attached to the leading end 104 of the endoscope 101, and is constituted by a second drive unit 110 for driving up and down, and the cell metamorphosing device 1 connected to the second drive unit 110. The first drive unit (not shown) and the second drive unit 110 are controlled by a drive controller 109. A capsule 111 containing a medium and nano-scale particles 45 is attached on the surface of the micro dishes 2 using an adhesive.

The treatment apparatus 100 is operated for the treatment as follows. The capsule 111 containing the nano-scale particles 45 is placed on the micro dishes 2 of the cell metamorphosing device 1, which is attached to the leading end 104 of the endoscope 101. The leading end 104 is inserted into the patient via his or her mouth. A position of a tumor or the like is confirmed with reference to images of an inner surface of a throat displayed on controller 103. The leading end 104 is moved toward the position of the tumor or the like under control of the drive controller 109, which then activates the second drive controller 110 of the treatment part 105. The capsule 111 on the micro dishes 2 of the cell metamorphosing device 1 is brought into contact with the tumor, and is blown out in order to expose the nano-scale particles 45. Thereafter, the nano-scale particles 45 will destroy the tumor as described above.

In the foregoing description, the cell metamorphosing device 1 is attached to the leading end 104 of the endoscope 101. Alternatively, the cell metamorphosing device 1 may be attached to a leading end of a catheter or the like. The catheter is inserted into a blood vessel, in which the nano-scale particles 45 are taken in, dosed or injected, so that harmful cells in the blood vessel can be destroyed.

(OTHER EXAMPLES)

Although the invention has been described with respect to some examples thereof, it will be understood that those skilled in the art that various modifications are possible without departing from the spirit of the present invention. For instance, the cell metamorphosing device 1 may include a microscope by which a destroyed state of a cell can be directly observed, or an electronic device such as a personal computer which can immediately acquire information of destroyed cells and can process the information into electronic data.

What is claimed is:

1. A cell metamorphosing method comprising:
mixing nano-scale particles in a medium containing harmful cells; and
applying vibration energy and thermal energy to the nano-scale particles, bombarding the nano-scale particles to the harmful cells, and destroying the harmful cells.

2. The method of claim 1 further comprising applying electric field energy to the nano-scale particles.

3. The method of claim 1, wherein the harmful cells are destroyed on a water-repellent film.

4. The method of claim 1, wherein the nano-scale particles are made of a material whose diameter is 10 nm to 100 nm.

5. A cell metamorphosing device comprising:
a medium container housing a mixed medium containing harmful cells and nano-scale particles;
a vibrator vibrating the mixed medium; and
a heater heating the mixed medium.

6. The device of claim 5, wherein the vibrator includes a conductive substrate placed apart from the medium container, and an AC bias is applied between the medium container and the substrate.

7. The device of claim 6, wherein the medium container is held by mechanical supports which are movable horizontally and vertically.

8. The device of claim 5, wherein the heater includes a diode provided in the medium container.

9. The device of claim 8, wherein the medium container is made of a silicon substrate, and the diode of the heater includes a cathode region and an anode region placed on the silicon substrate.

10. The device of claim 5 further comprising a magnetic field applying unit which applies a magnetic field to the mixed medium.

11. The device of claim 10, wherein the magnetic field applying unit is constituted by an inductor wrapped around the medium holder.

12. The device of claim 6 further comprising a magnetic field applying unit which applies a magnetic field to the mixed medium.

13. The device of claim 12, wherein the magnetic field applying unit is constituted by an inductor wrapped around the medium holder.

14. The device of claim 5 further comprising an electromagnetic wave applying unit which applies electromagnetic waves to the mixed medium.

15. The device of claim 5, wherein a water-repellent film extends over the surface of the medium container.

16. A treatment apparatus comprising:
an endoscope;
a light source supplying light to the endoscope;
a first drive unit coupled to the endoscope and activating a leading end of the endoscope; and
a treatment section attached to the leading end of the endoscope, and including a cell metamorphosing device and a second drive unit, the cell metamorphosing device having a medium container housing a medium containing harmful cells and nano-scale particles, a vibrator applying vibrations to the mixed medium, and a heater heating the mixed medium, and the second drive unit moving the cell metamorphosing device in an imaging direction of the endoscope.
